(19) United States
(12) Patent Application Publication
(10) Pub. No.: US 2005/0089912 A1
(43) Pub. Date: Apr. 28, 2005

(54) Alternatively spliced isoforms of nuclear factor kappa-B, subunit 1 (NFKB1)

(76) Inventors: Christopher D. Armour, Kirkland, WA (US); John C. Castle, Seattle, WA (US); Philip W. Garrett-Engele, Seattle, WA (US); Zhengyan Kan, Bellevue, WA (US); Christopher K. Raymond, Seattle, WA (US); Nicholas F. Tsinoremas, Sammamish, WA (US)

Correspondence Address: R. Douglas Bradley;Merck & Co., Inc., P.O. Box 2000, Patent Department RY60-30, Rahway, NJ, 07065-0907, US

(21) Appl. No.: 10/960,409

(22) Filed: Oct. 6, 2004

Related U.S. Application Data
(60) Provisional application No. 60/509,361, filed on Oct. 7, 2003.

(51) Int. Cl. C12Q 1/68; G01N 33/53; C07K 14/72; C07K 16/18

(52) U.S. Cl. 435/6; 514/12; 435/7.1; 435/69.1; 435/320.1; 435/325; 530/358; 530/388.1; 536/23.5

(57) ABSTRACT

The present invention features nucleic acids and polypeptides encoding two novel splice variant isoforms of nuclear factor kappa-B, subunit 1 (NFKB1). The polynucleotide sequences of NFKB1sv1 and NFKB1sv2 are provided by SEQ ID NO 3 and SEQ ID NO 5, respectively. The amino acid sequences for NFKB1sv1 and NFKB1sv2 are provided by SEQ ID NO 4 and SEQ ID NO 6, respectively. The present invention also provides methods for using NFKB1sv1 and NFKB1sv2 polynucleotides and proteins to screen for compounds that bind to NFKB1sv1 and NFKB1sv2, respectively.

This application claims priority to U.S. Provisional Patent Application Serial No. 60/509,361 filed on Oct. 7, 2003, which is incorporated by reference herein in its entirety.

### BACKGROUND OF THE INVENTION

The references cited herein are not admitted to be prior art to the claimed invention.

The transcription factor nuclear factor kappa-B (NFκB) plays an integral role in the cellular response to a wide array of harmful stimuli, including cytokines, bacterial lipopolysaccharide, viral infection, phorbol esters, UV radiation, and free radicals. NFκB regulates genes involved in immune function, inflammation responses, growth control, cell death, cell adhesion, and viral replication (for reviews see Baldwin, A. S., 1996, Annu. Rev. Immunol. 14, 649-681; Baeuerle, P. A. & Baltimore, D., 1996, Cell 87, 13-20; Stancovski, I. & Baltimore, D., 1997 Cell, 91, 299-302). The function of NFκB has been implicated in diseases as varied as rheumatoid arthritis, lupus, HIV-AIDS, influenza, septic shock, atherosclerosis; oncogenesis, and apoptosis (Baldwin, 1996).

The mammalian NFκB family of inducible transcription factors is comprised of five structurally related polypeptides: NFKB1 (p50/p105), NFKB2 (p52/p100), p65 (RelA), RelB, and c-Rel (reviewed in Finco and Baldwin, 1995, Immunity 3: 263-72; Baldwin, 1996, Annu Rev Immunol 14: 649-83; Chen et al., 1999, Clinical Chemistry 45: 7-17). All of the NFκB family members share a highly conserved Rel homology domain composed of about 300 amino acids that is responsible for DNA binding, dimerization, and interactions with IκB proteins, a family of NFκB inhibitors (reviewed in Baldwin, 1996, Annu Rev Immunol 14: 649-83; Chen et al., 1999, Clinical Chemistry 45: 7-17). NFκB family member polypeptides associate to form transcriptionally competent homo- and heterodimers including p50/p50, p52/p52, RelA/RelA, RelA/c-Rel, and p50/p65 (RelA) (reviewed in Baldwin, 1996, Annu Rev Immunol 14: 649-83; Chen et al., 1999, Clinical Chemistry 45: 7-17). Of these dimers, p50 (NFKB1)/p65 (RelA), the prototypical NFKB heterodimer, is most abundant and has been most intensively studied.

The human NFKB1 gene encodes two functionally distinct proteins termed p50 and p105 (Ghosh et al., 1990, Cell 62: 1019-1029; Kieran et al., 1990, Cell 62: 1007-1018; Heron et al., 1995, Genomics 30: 493-505). The p50 protein corresponds to the N-terminus of the p105 protein and, upon binding to the p65 (RelA) protein, forms the prototypical NFκB transcription factor complex. In contrast, the p105 NFKB1 protein functions as a Rel-specific inhibitor (IκB) (Rice et al., 1992, Cell 71: 243-253). p50 is generated cotranslationally by a proteasome-mediated process that ensures the production of both p50 and p105 and preserves their independent functions (Lin et al., 1998, Cell 92: 819-828). Cotranslational folding of the N-terminal portion of p105 protects the amino acid region that forms p50 from degradation by the proteasome, whereas folding of sequences downstream of a glycine rich amino acid region precludes protein processing altogether, leading to the production of p105 (Lin et al., 1998).

The NFKB1 gene has 24 exons and several functionally important domains (Heron et al., 1995, Genomics 30: 493-505). Exons 4 through 11 encode most of the Rel homology domain that is required for DNA binding, dimerization, and interaction with IκB inhibitors (Heron et al, 1995; reviewed in Baldwin, 1996, Annu Rev Immunol 14: 649-83; Chen et al., 1999, Clinical Chemistry 45: 7-17). Exon 12 includes a nuclear localization signal (NLS) and a glycine rich region (Heron et al., 1995). The glycine rich region in exon 12 (amino acids 375-401) is required for the generation of p50 in COS cells (Lin and Ghosh, 1996, Mol. Cell Biol. 16: 2248-2254). Seven complete and one partial ankyrin repeat motifs are encoded by exons 15 to 21 (Heron et al., 1995). Ankyrin repeats are also found in the IκB family of inhibitor polypeptides. The ankyrin repeats in the p105 polypeptide allow p105 to bind and sequester other Rel-related proteins in the cytoplasm, thereby functioning as a Rel-specific inhibitor (IκB) (Rice et al., 1992, Cell 71: 243-253). The carboxy-terminus of p50 is between amino acid 413 and amino acid 436 (approximately amino acid 433); this region is encoded by exons 13 and 14 (Heron et al., 1995; Lin et al., 1998).

In cells that have not been appropriately stimulated, NFκB is located in the cytoplasm and is bound to an IκB protein, an inhibitor of NFκB. Structural analysis of human or murine p50/p65 NFκB complexed with IκBα indicates that IκBα binding occludes critical amino acid residues required for DNA binding (Huxford et al., 1998, Cell 95: 759-770; Jacobs and Harrison, 1998, Cell 95: 749-758). Furthermore, amino acid residues immediately preceding the nuclear localization signals of both NFκB p50 and p65 subunits are tethered to the IκBα amino-terminal ankyrin repeats, impeding NFκB from nuclear import machinery recognition (Huxford et al., 1998; Jacobs and Harrison, 1998). In response to cellular stimuli, the IκB protein inhibitors are phosphorylated by the IκB kinase (IKK) complex leading to their rapid ubiquitination and subsequent degradation by the 26S proteasome (Chen et al., 1995, Genes Dev 9:1586-1597; DiDonata et al., 1997, Nature 388: 548-554; Zandi et al., 1997, Cell 91: 243-252; reviewed in Baldwin, 1996, Annu Rev Immunol 14: 649-83; Chen et al., 1999, Clinical Chemistry 45: 7-17; Baldwin, 2001, J Clin Investig 107: 241-246; Barnes and Karin, 1997, New England J of Med 336: 1066-1071). Degradation of IκB inhibitors releases NFκB, allowing this transcription factor to accumulate in the nucleus where it binds to specific sequences (κB recognition sequences) in the promoter regions of target genes (Stancovski and Baltimore, 1997, Cell 91: 299-302; reviewed in Baldwin, 1996, Annu Rev Immunol 14: 649-83; Chen et al., 1999, Clinical Chemistry 45: 7-17; Baldwin, 2001, J. Clin Investig 107: 241-246; Barnes and Karin, 1997, New England J of Med 336: 1066-1071). In addition to signals that promote its accumulation in the nucleus, NFκB transcriptional activity is controlled by phophorylation of its subunits (Zhong et al., 1998, Mol. Cell 1: 661-671). Furthermore, because the IκB genes have κB recognition sequences in their promoter regions, NFκB induces the synthesis of IκB genes, which enter the nucleus to transport NFκB to the cytoplasm, thereby terminating the activation of gene expression (reviewed in Barnes and Karin, 1997).

NFκB can be activated by exposure of cells to bacterial lipopolysaccharide, inflammatory cytokines such as tumor necrosis factor-α or interleukin-1β, viral infection or expression of certain viral gene products, ultraviolet radiation, phorbol esters, hydrogen peroxide, and B- or T-cell activation (reviewed in Barnes and Karin, 1997; Baldwin, 1996; Chen et al., 1999; Baldwin, 2001). NFKB has a clear role in the inducible regulation of a wide variety of genes involved apoptosis and cellular proliferation, genes involved in immune function and inflammation responses (GM-CSF, interleukin-6, interleukin-8, interleukin-2); genes involved in cell adhesion (VCAM-1, ICAM-1, Mad-CAM-1, and E-selectin); the peptide transporter TAP-1, the proteasome subunit LMP2, and the MIC class II variant chain gene (reviewed in Baldwin, 1996; Barnes and Karin, 1997).

Consistent with the function of NFκB in inducing expression of genes involved in apoptosis, cellular proliferation, inflammation and immune response, NFκB has been implicated in a variety of human diseases and disorders. For example, NFκB is activated in arthritic synovium, and therapies that are used for treatment of rheumatoid arthritis are now known to block NFκB (Yang et al., 1995, FEBS Letts. 361: 89-96; reviewed in Baldwin, 1996). Furthermore, cyclooxygenase-2, an inducible enzyme regulated by NFκB, is responsible for the increased production of prostaglandins and thromboxane in inflammatory disease (Yamamoto et al., 1995, J Biol Chem 270: 31315-31350; Crofford et al., 1994, J Clin Invest 93: 1095-1101; reviewed in Barnes and Karin, 1997). Because NFκB is activated by many of the factors that increase the inflammatory response and because NFκB activation leads to the coordinated expression of many genes that encode proteins involved in the amplification and perpetuation of the inflammatory response, NFκB is a target for new types of anti-inflammatory treatment (reviewed in Barnes and Karin, 1997; Yamamoto and Gaynor, 2001, J Clin Invest 107: 135-142).

NFκB has also been implicated in oncogenesis (reviewed in Chen et al., 1999; Baldwin, 2001). Exposure of cells to low oxygen concentrations results in the activation of NFκB which is likely to be important in angiogenesis associated with tumorigenesis (Koong et al., 1994, Cancer Res 54: 1425-1430; reviewed in Baldwin, 1996). Activation of NFκB is required in oncogenic Ras-induced cell transformation (Mayo et al., 1997, Science 278: 1812-1815). The expression or activation of NFκB is evident in several human cancers, including breast cancer (Sovak et al., 1997, J Clin Invest 100: 2952-2960; Nakshatri et al., 1997, Mol Cell Biol 17: 3629-3639), non-small cell lung carcinoma (Mukhopadhyay et al., 1995, Oncogene 11: 999-1003), thyroid cancer (Gilmore et al., 1996, Oncogene 13: 1367-1378), T- or B-cell lymphocyte leukemia (Bargou et al., 1996, Blood 87: 4340-4347), and several virally induced tumors (Miwa et al., 1997, Leukemia 11(Suppl 3): 65-66; Berger et al., 1997, Lymphoma 26:239-250; Blumberg, 1997, Res Virol 148: 91-94; Nasti et al., 1997, Biomed Pharmacother 51: 243-251). Furthermore, the constitutive nuclear localization of NFKB was found to be required for sustained proliferation of Hodgkin lymphoma and was considered to be a potential diagnostic marker (Bargou et al., 1996; Bargou et al., 1997, J Clin Invest 100: 2961-2969).

NFκB contributes to both pro-apoptotic and anti-apoptotic pathways (reviewed in Chen et al, 1999; Baldwin, 2001; Yamamoto and Gaynor, 2001, J. Clin Invest 107: 135-142). NFκB transmits two signals. One is required for the induction of the pro-apoptotic proto-oncogene c-myc, and the second is an anti-apoptotic signal that counterbalances c-myc cytotoxicity (Romashkova and Makarov, 1999, Nature 401: 86-90). In addition, induction of p53 causes activation of NFκB that correlates with the ability of p53 to induce apoptosis, indicating that NFκB is essential for p53 mediated cell death (Ryan et al., 2000, Nature 404: 892-897).

Viruses have evolved strategies to modulate NFκB activity to enhance viral replication, host cell survival, and evasion of the immune response. For example, two NFκB binding sites in the HIV-1 long terminal repeat mediate inducible gene expression (Roulston et al., 1995, Microbiol Rev 59: 481-505; reviewed in Hiscott et al, 2001, J Clin Invest 107: 143-151). Persistent activation of the NFKB pathway may lead to oncogenic transformation (Rayet and Gelinas, 1999, Oncogene 18: 6938-6947).

Although mice lacking the p50/p105 (NFKB1) subunits develop normally, they exhibit defects in immune responses (Sha et al., 1995, Cell 80: 321-330). Production of antibodies in these mice is impaired and B cells fail to proliferate in response to lipopolysaccharide (Sha et al., 1995). Total serum Ig was four-fold lower in mice lacking NFκB1 and IgE was reduced approximately forty-fold, indicating that p50 plays a role in heavy-chain class switching (Sha et al., 1995; reviewed in Baldwin, 1996).

Several compounds and proteins have been identified that inhibit NFκB. The IκB proteins bind to and inhibit NFκB translocation into the nucleus (reviewed in Baldwin, 1996; Baldwin, 2001). Tepoxalin, a dual inhibitor of cyclooxygenase and 5-lipoxygenase, also functions to inhibit NFκB in multiple cell types (Kazmi et al., 1995, J. Cell. Biochem. 57: 299-310). Interleukin-10 has been reported to inhibit the induction of NFκB by lipopolysaccharide in human peripheral blood mononuclear cells (Wang et al., 1995, J. Biol Chem 270: 9558-9563). Glucocorticoids, nitric oxide, cyclosporine, rapamycin, and salicylates also act as inhibitors of NFκB activation (reviewed in Baldwin, 1996, Chen et al., 1999; Baldwin, 2001; Yamamoto et al., 2001).

Because of the multiple therapeutic values of drugs targeting the NFκB pathway, and the essential regulatory role played by NFKB1, there is a need in the art to identify new isoform variants of NFKB1 and for compounds that selectively bind to isoforms of NFKB1. The present invention is directed towards two novel NFKB1 isoforms (NFKB1sv1 and NFKB1sv2) and uses thereof.

### BRIEF DESCRIPTION OF THE FIGURES

FIG. 1A illustrates the exon structure of NFKB1 mRNA corresponding to the known long reference form of NFKB1 mRNA (labeled NM_{—}003998.2) and the exon structure corresponding to the inventive short form splice variants (labeled NFKB1sv1 and NFKB1sv2). FIG. 1B depicts the nucleotide sequences of the exon junctions resulting from the splicing of exon 4 to exon 6 (SEQ ID NO 1) in the case of NFKB1sv1 mRNA and the splicing of exon 16 to exon 19 (SEQ ID NO 2) in the case of NFKB1sv2 mRNA. In FIG. 1B, in the case of NFKB1sv1, the nucleotides shown in italics represent the 20 nucleotides at the 3′ end of exon 4 and the nucleotides shown in underline represent the 20 nucleotides at the 5′ end of exon 6 and in the case of NFKB1sv2, the nucleotides shown in italics represent the 20 nucleotides at the 3′ end of exon 16 and the nucleotides shown in underline represent the 20 nucleotides at the 5′ end of exon 19.

### SUMMARY OF THE INVENTION

Microarray experiments and RT-PCR have been used to identify and confirm the presence of novel splice variants of human NFKB1 mRNA. More specifically, the present invention features polynucleotides encoding different protein isoforms of NFKB1. A polynucleotide sequence encoding NFKB1sv1 is provided by SEQ ID NO 3. An amino acid sequence for NFKB1sv1 is provided by SEQ ID NO 4. A polynucleotide sequence encoding NFKB1sv2 is provided by SEQ ID NO 5. An amino acid sequence for NFKB1sv2 is provided by SEQ ID NO 6.

Thus, a first aspect of the present invention describes a purified NFKB1sv1 encoding nucleic acid and a purified NFKB1sv2 encoding nucleic acid. The NFKB1sv1 encoding nucleic acid comprises SEQ ID NO 3 or the complement thereof. The NFKB1sv2 encoding nucleic acid comprises SEQ ID NO 5 or the complement thereof. Reference to the presence of one region does not indicate that another region is not present. For example, in different embodiments the inventive nucleic acid can comprise, consist, or consist essentially of an encoding nucleic acid sequence of SEQ ID NO 3 or alternatively can comprise, consist, or consist essentially of the nucleic acid sequence of SEQ ID NO 5.

Another aspect of the present invention describes a purified NFKB1sv1 polypeptide that can comprise, consist or consist essentially of the amino acid sequence of SEQ ID NO 4. An additional aspect describes a purified NFKB1sv2 polypeptide that can comprise, consist, or consist essentially of the amino acid sequence of SEQ ID NO 6.

Another aspect of the present invention describes expression vectors. In one embodiment of the invention, the inventive expression vector comprises a nucleotide sequence encoding a polypeptide comprising, consisting, or consisting essentially of SEQ ID NO 4, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 6, wherein the nucleotide sequence is transcriptionally coupled to an exogenous promoter.

Alternatively, the nucleotide sequence comprises, consists, or consists essentially of SEQ ID NO 3, and is transcriptionally coupled to an exogenous promoter. In another embodiment, the nucleotide sequence comprises, consists, or consists essentially of SEQ ID NO 5, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 3 or SEQ ID NO 5, or a nucleotide sequence encoding a polypeptide comprising, consisting, or consisting essentially of an amino acid sequence of SEQ ID NO 4 or SEQ ID NO 6, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing NFKB1sv1 or NFKB1sv2 polypeptide comprising SEQ ID NO 4 or SEQ ID NO 6, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to NFKB1sv1 as compared to one or more NFKB1 isoform polypeptides that are not NFKB1sv1. In another embodiment, a purified antibody preparation is provided comprising antibody that binds selectively to NFKB1sv2 as compared to one or more NFKB1 isoform polypeptides that are not NFKB1sv2.

Another aspect of the present invention provides a method of screening for a compound that binds to NFKB1sv1, NFKB1sv2 or fragments thereof. In one embodiment, the method comprises the steps of: (a) expressing a polypeptide comprising the amino acid sequence of SEQ ID NO 4 or a fragment thereof from recombinant nucleic acid; (b) providing to said polypeptide a labeled NFKB1 ligand that binds to said polypeptide and a test preparation comprising one or more test compounds; (c) and measuring the effect of said test preparation on binding of said test preparation to said polypeptide comprising SEQ ID NO 4. Alternatively, this method could be performed using SEQ ID NO 6 instead of SEQ ID NO 4.

In another embodiment of the method, a compound is identified that binds selectively to NFKB1sv1 polypeptide as compared to one or more NFKB1 isoform polypeptides that are not NFKB1sv1. This method comprises the steps of: providing a NFKB1sv1 polypeptide comprising SEQ ID NO 4; providing a NFKB1 isoform polypeptide that is not NFKB1sv1; contacting said NFKB1sv1 polypeptide and said NFKB1 isoform polypeptide that is not NFKB1sv1 with a test preparation comprising one or more test compounds; and determining the binding of said test preparation to said NFKB1sv1 polypeptide and to NFKB1 isoform polypeptide that is not NFKB1sv1, wherein a test preparation that binds to said NFKB1sv1 polypeptide but does not bind to said NFKB1 isoform polypeptide that is not NFKB1sv1 contains a compound that selectively binds said NFKB1sv1 polypeptide. Alternatively, the same method can be performed using NFKB1sv2 polypeptide comprising, consisting, or consisting essentially of SEQ ID NO 6.

In another embodiment of the invention, a method is provided for screening for a compound able to bind to or interact with a NFKB1sv1 protein or a fragment thereof comprising the steps of: expressing a NFKB1sv1 polypeptide comprising SEQ ID NO 4 or a fragment thereof from a recombinant nucleic acid; providing to said polypeptide a labeled NFKB1 ligand that binds to said polypeptide and a test preparation comprising one or more compounds; and measuring the effect of said test preparation on binding of said labeled NFKB1 ligand to said polypeptide, wherein a test preparation that alters the binding of said labeled NFKB1 ligand to said polypeptide contains a compound that binds to or interacts with said polypeptide. In an alternative embodiment, the method is performed using NFKB1sv2 polypeptide comprising, consisting, or consisting essentially of SEQ ID NO 6 or a fragment thereof.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “NFKB1” refers to human nuclear factor kappa-B, subunit 1 protein (NP_{—}003989). In contrast, reference to a NFKB1 isoform, includes NP_{—}003989 and other polypeptide isoform variants of NFKB1.

As used herein, “NFKB1sv1” and “NFKB1sv2” refer to splice variant isoforms of human NFKB1 protein, wherein the splice variants have the amino acid sequence set forth in SEQ ID NO 4 (for NFKB1sv1) and SEQ ID NO 6 (for NFKB1sv2).

As used herein, “NFKB1” refers to polynucleotides encoding NFKB1.

As used herein, “NFKB1sv1” refers to polynucleotides encoding NFKB1sv1 having an amino acid sequence set forth in SEQ ID NO 4. As used herein, “NFKB1sv2” refers to polynucleotides encoding NFKB1sv2 having an amino acid sequence set forth in SEQ ID NO 6.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature -where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human NFKB1sv1 and NFKB1sv2 that are alternatively spliced isoforms of NFKB1, and to the amino acid sequences encoding these proteins. SEQ ID NO 3 and SEQ ID NO 5 are polynucleotide sequences representing exemplary open reading frames that encode the NFKB1sv1 and NFKB1sv2 proteins, respectively. SEQ ID NO 4 shows the polypeptide sequence of NFKB1sv1. SEQ ID NO 6 shows the polypeptide sequence of NFKB1sv2.

NFKB1sv1 and NFKB1sv2 polynucleotide sequences encoding NFKB1sv1 and NFKB1sv2 proteins, as exemplified and enabled herein include a number of specific, substantial and credible utilities. For example, NFKB1sv1 and NFKB1sv2 encoding nucleic acids were identified in an mRNA sample obtained from a human source (see Example 1). Such nucleic acids can be used as hybridization probes to distinguish between cells that produce NFKB1sv1 and NFKB1sv2 transcripts from human or non-human cells (including bacteria) that do not produce such transcripts. Similarly, antibodies specific for NFKB1sv1 or NFKB1sv2 can be used to distinguish between cells that express NFKB1sv1 or NFKB1sv2 from human or non-human cells (including bacteria) that do not express NFKB1sv1 or NFKB1sv2.

NFKB1 is an important drug target for the management of immune function and inflammation responses and apoptosis, as well as diseases such as rheumatoid arthritis, lupus, HIV-AIDS, influenza, and cancer (reviewed in Baldwin, A. S., 1996; Chen et al., 1999, Baldwin, 2001; Yamamoto et al., 2001; Hiscott et al., 2001). Given the potential importance of NFKB1 activity to the therapeutic management of a wide array of diseases, it is of value to identify NFKB1 isoforms and identify NFKB1-ligand compounds that are isoform specific, as well as compounds that are effective ligands for two or more different NFKB1 isoforms. In particular, it may be important to identify compounds that are effective inhibitors of a specific NFKB1 isoform activity, yet do not bind to or interact with a plurality of different NFKB1 isoforms. Compounds that bind to or interact with multiple NFKB1 isoforms may require higher drug doses to saturate multiple NFKB1-isoform binding sites and thereby result in a greater likelihood of secondary non-therapeutic side effects. Furthermore, biological effects could also be caused by the interaction of a drug with the NFKB1sv1 or NFKB1sv2 isoforms specifically. For the foregoing reasons, NFKB1sv1 and NFKB1sv2 proteins represent useful compound binding targets and have utility in the identification of new NFKB1-ligands exhibiting a preferred specificity profile and having greater efficacy for their intended use.

In some embodiments, NFKB1sv1 and NFKB1sv2 activity is modulated by a ligand compound to achieve one or more of the following: prevent or reduce the risk of occurrence, or recurrence of diseases resulting from inflammatory or apoptotic cellular responses, rheumatoid arthritis, septic shock, lupus, HIV-AIDS, viral infections, and cancer. Compounds that treat cancers are particularly important because of the cause-and-effect relationship between cancers and mortality (National Cancer Institute's Cancer Mortality Rates Registry, http://www3.cancer.gov/atlasplus/charts.html, last visited Sep. 24, 2003).

Compounds modulating NFKB1sv1 or NFKB1sv2 include agonists, antagonists, and allosteric modulators. Inhibitors of NFKB1 achieve clinical efficacy by a number of known and unknown mechanisms. While not wishing to be limited to any particular theory of therapeutic efficacy, generally, but not always, NFKB1sv1 or NFKB1sv2 compounds will be used to inhibit release of NF-kappa-B to the nucleus or transcriptional activation of NF-kappa-B once it relocates to the nucleus. Therefore, agents that modulate NFKB1 activity may be used to achieve a therapeutic benefit for any disease or condition due to, or exacerbated by, abnormal levels of NF-kappa-B protein or its activity.

NFKB1sv1 or NFKB1sv2 activity can also be affected by modulating the cellular abundance of transcripts encoding NFKB1sv1 or NFKB1sv2, respectively. Compounds modulating the abundance of transcripts encoding NFKB1sv1 or NFKB1sv2 include a cloned polynucleotide encoding NFKB1sv1 or NFKB1sv2, respectively, that can express NFKB1sv1 or NFKB1sv2 in vivo, antisense nucleic acids targeted to NFKB1sv1 or NFKB1sv2 transcripts, and enzymatic nucleic acids, such as ribozymes and RNAi, targeted to NFKB1sv1 or NFKB1sv2 transcripts.

In some embodiments, NFKB1sv1 or NFKB1sv2 activity is modulated to achieve a therapeutic effect upon diseases in which regulation of NF-kappa-B is desirable. For example, rheumatoid arthritis and lupus may be treated by modulating NFKB1sv1 or NFKB1sv2 activities to inhibit activation of genes involved in inflammation and immune responses. In other embodiments, HIV-AIDS and other viral infections may be treated by inhibiting the activation of genes involved in viral replication. In other embodiments, cancer may be treated by modulating NFKB1sv1 or NFKB1sv2 to inhibit genes involved in oncogenesis. In other embodiments, disorders resulting from either high or low levels of apoptosis may be treated by modulating NFKB1sv1 or NFKBsv2 to either inhibit or induce genes involved in apoptosis.

NFKB1sv1 and NFKB1sv2 Nucleic Acids

NFKB1sv1 nucleic acids contain regions that encode for polypeptides comprising, consisting, or consisting essentially of SEQ ID NO 4. NFKB1sv2 nucleic acids contain regions that encode for polypeptides comprising, consisting, or consisting essentially of SEQ ID NO 6. The NFKB1sv1 and NFKB1sv2 nucleic acids have a variety of uses, such as use as a hybridization probe or PCR primer to identify the presence of NFKB1sv1 or NFKB1sv2 nucleic acids, respectively; use as a hybridization probe or PCR primer to identify nucleic acids encoding for proteins related to NFKB1sv1 or NFKB1sv2, respectively; and/or use for recombinant expression of NFKB1sv1 or NFKB1sv2 polypeptides, respectively. In particular, NFKB1sv1 polynucleotides do not have the polynucleotide region that consists of exon 5 of the NFKB1 gene. NFKB1sv2 polynucleotides do not have the polynucleotide region that consists of exons 17 and 18 of the NFKB1 gene.

Regions in NFKB1sv1 or NFKB1sv2 nucleic acid that do not encode for NFKB1sv1 or NFKB1sv2, or are not found in SEQ ID NO 3 or SEQ ID NO 5, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding NFKB1sv1 or NFKB1sv2 related proteins from different sources. Obtaining nucleic acids NFKB1sv1 or NFKB1sv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

NFKB1sv1 or NFKB1sv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

NFKB1sv1 and NFKB1sv2 Probes

Probes for NFKB1sv1 or NFKB1sv2 contain a region that can specifically hybridize to NFKB1sv1 or NFKB1sv2 target nucleic acids, respectively, under appropriate hybridization conditions and can distinguish NFKB1sv1 or NFKB1sv2 nucleic acids from each other and from non-target nucleic acids, in particular NFKB1 polynucleotides containing exon 5, or exons 17 and 18. Probes for NFKB1sv1 or NFKB1sv2 can also contain nucleic acid regions that are not complementary to NFKB1sv1 or NFKB1sv2 nucleic acids.

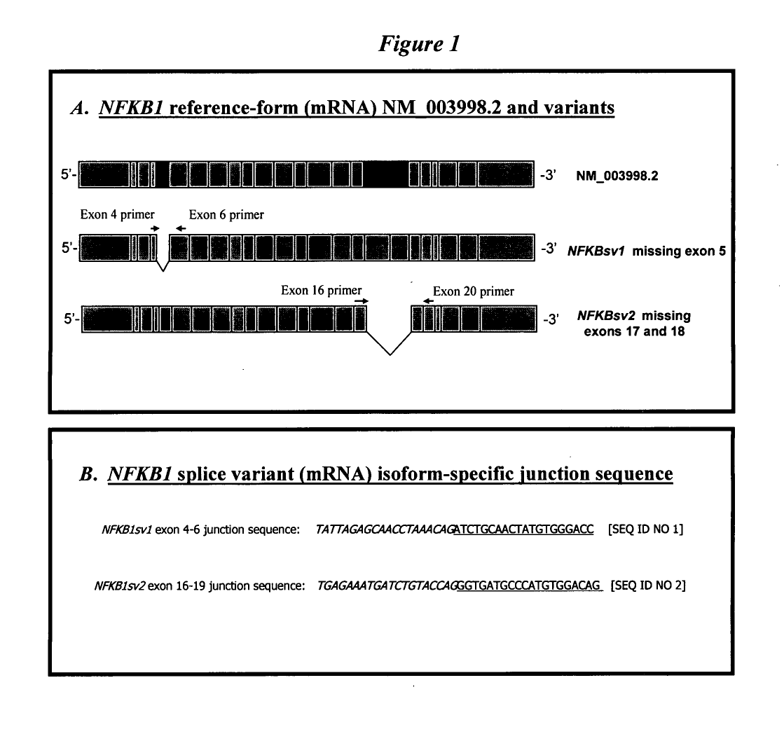

In embodiments where, for example, NFKB1sv1 or NFKB1 sv2 polynucleotide probes are used in hybridization assays to specifically detect the presence of NFKB1sv1 or NFKB1sv2 polynucleotides in samples, the NFKB1sv1 or NFKB1sv2 polynucleotides comprise at least 20 nucleotides of the NFKB1sv1 or NFKB1sv2 sequence that correspond to the respective novel exon junction polynucleotide regions. In particular, for detection of NFKB1sv1, the probe comprises at least 20 nucleotides of the NFKB1sv1 sequence that corresponds to an exon junction polynucleotide created by the alternative splicing of exon 4 to exon 6 of the primary transcript of the NFKB1 gene (see FIGS. 1A and 1B). For example, the polynucleotide sequence: 5′ ACCTAAACAGATCTGCAACT 3′ [SEQ ID NO 7] represents one embodiment of such an inventive NFKB1sv1 polynucleotide wherein a first 10 nucleotide region is complementary and hybridizable to the 3′ end of exon 4 of the NFKB1 gene and a second 10 nucleotide region is complementary and hybridizable to the 5′ end of exon 6 of the NFKB1 gene (see FIG. 1B).

In another embodiment, for detection of NFKB1sv2, the probe comprises at least 20 nucleotides of the NFKB1sv2 sequence that corresponds to an exon junction polynucleotide created by the alternative splicing of exon 16 to exon 19 of the primary transcript of the NFKB1 gene (see FIGS. 1A and 1B). For example, the polynucleotide sequence: 5′ TCTGTACCAGGGTGATGCCC 3′ [SEQ ID NO 8] represents one embodiment of such an inventive NFKB1sv2 polynucleotide wherein a first 10 nucleotides region is complementary and hybridizable to the 3′ end of exon 16 of the NFKB1 gene and a second 10 nucleotide region is complementary and hybridizable to the 5′ end of exon 19 of the NFKB1 gene (see FIG. 1B).

In some embodiments, the first 20 nucleotides of an NFKB1sv1 probe comprise a first continuous region of 5 to 15 nucleotides that is complementary and hybridizable to the 3′ end of exon 4 and a second continuous region of 5 to 15 nucleotides that is complementary and hybridizable to the 5′ end of exon 6. In some embodiments, the first 20 nucleotides of an NFKB1sv2 probe comprise a first continuous region of 5 to 15 nucleotides that is complementary and hybridizable to the 3′ end of exon 16 and a second continuous region of 5 to 15 nucleotides that is complementary and hybridizable to the 5′ end of exon 19.

In other embodiments, the NFKB1 sv1 or NFKB1sv2 polynucleotide comprises at least 40, 60, 80 or 100 nucleotides of the NFKB1sv1 or NFKB1sv2 sequence, respectively, that correspond to a junction polynucleotide region created by the alternative splicing of exon 4 to exon 6 in the case of NFKB1sv1 or in the case of NFKB1sv2, by the alternative splicing of exon 16 to exon 19 of the primary transcript of the NFKB1 gene. In embodiments involving NFKB1sv1, the NFKB1sv1 polynucleotide is selected to comprise a first continuous region of at least 5 to 15 nucleotides that is complementary and hybridizable to the 3′ end of exon 4 and a second continuous region of at least 5 to 15 nucleotides that is complementary and hybridizable to the 5′ end of exon 6. Similarly, in embodiments involving NFKB1sv2, the NFKB1sv2 polynucleotide is selected to comprise a first continuous region of at least 5 to 15 nucleotides that is complementary and hybridizable to the 3′ end of exon 16 and a second continuous region of at least 5 to 15 nucleotides that is complementary and hybridizable to the 5′ end of exon 19. As will be apparent to a person of skill in the art, a large number of different polynucleotide sequences from the region of the exon 4 to exon 6 splice junction and the exon 16 to exon 19 splice junction may be selected which will, under appropriate hybridization conditions, have the capacity to detectably hybridize to NFKB1sv1 or NFKB1sv2 polynucleotides, respectively, and yet will hybridize to a much less extent or not at all to NFKB1 isoform polynucleotides wherein exon 4 is not spliced to exon 6 or wherein exon 16 is not spliced to exon 19, respectively.

Preferably, non-complementary nucleic acid that is present has a particular purpose such as being a reporter sequence or being a capture sequence. However, additional nucleic acid need not have a particular purpose as long as the additional nucleic acid does not prevent the NFKB1sv1 or NFKB1sv2 nucleic acid from distinguishing between target polynucleotides, e.g., NFKB1sv1 or NFKB1sv2 polynucleotides, and non-target polynucleotides, including, but not limited to NFKB1 polynucleotides not comprising the exon 4 to exon 6 splice junction or the exon 16 to exon 19 splice junctions found in NFKB1sv1 or NFKB1sv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular nd Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C in buffer composed of 6×SSC, 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

NFKB1sv1 or NFKB1sv2 polynucleotides, such as those comprising SEQ ID NO 3 or SEQ ID NO 5, respectively, can be used to make NFKB1sv1 or NFKB1sv2 polypeptides, respectively. In particular, NFKB1sv1 or NFKB1sv2 polypeptides can be expressed from recombinant nucleic acids in a suitable host or in vitro using a translation system. Recombinantly expressed NFKB1sv1 or NFKB1sv2 polypeptides can be used, for example, in assays to screen for compounds that bind NFKB1sv1 or NFKB1sv2, respectively. Alternatively, NFKB1sv1 or NFKB1sv2 polypeptides can also be used to screen for compounds that bind to one or more NFKB1 isoforms, but do not bind to NFKB1sv1 or NFKB1sv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TKW) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 3 or SEQ ID NO 5 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

NFKB1sv1 and NFKB1sv2 Polypeptides

NFKB1sv1 polypeptides contain an amino acid sequence comprising, consisting or consisting essentially of SEQ ID NO 4. NFKB1sv2 polypeptides contain an amino acid sequence comprising, consisting or consisting essentially of SEQ ID NO 6. NFKB1sv1 or NFKB1sv2 polypeptides have a variety of uses, such as providing a marker for the presence of NFKB1sv1 or NFKB1sv2, respectively; use as an immunogen to produce antibodies binding to NFKB1sv1 or NFKB1sv2, respectively; use as a target to identify compounds binding selectively to NFKB1sv1 or NFKB1sv2, respectively; or use in an assay to identify compounds that bind to one or more isoforms of NFKB1 but do not bind to or interact with NFKB1sv1 or NFKB1sv2, respectively.

In chimeric polypeptides containing one or more regions from NFKB1sv1 or NFKB1sv2 and one or more regions not from NFKB1sv1 or NFKB1sv2, respectively, the region(s) not from NFKB1sv1 or NFKB1sv2, respectively, can be used, for example, to achieve a particular purpose or to produce a polypeptide that can substitute for NFKB1sv1 or NFKB1sv2, or fragments thereof. Particular purposes that can be achieved using chimeric NFKB1sv1 or NFKB1sv2 polypeptides include providing a marker for NFKB1sv1 or NFKB1sv2 activity, respectively, enhancing an immune response, and modulating the levels of NF-kappa-B in the nucleus or the activity of NF-kappa-B.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional NFKB1sv1 and NFKB1sv2

Functional NFKB1sv1 and NFKB1sv2 are different protein isoforms of NFKB1. The identification of the amino acid and nucleic acid sequences of NFKB1sv1 or NFKB1sv2 provide tools for obtaining functional proteins related to NFKB1sv1 or NFKB1sv2, respectively, from other sources, for producing NFKB1sv1 or NFKB1sv2 chimeric proteins, and for producing functional derivatives of SEQ ID NO 4 or SEQ ID NO 6.

NFKB1sv1 or NFKB1sv2 polypeptides can be readily identified and obtained based on their sequence similarity to NFKB1sv1 (SEQ ID NO 4) or NFKB1sv2 (SEQ ID NO 6), respectively. In particular, NFKB1sv1 lacks a 99 base pair region corresponding to exon 5 of the NFKB1 gene. The deletion of exon 5 does not disrupt the protein reading frame as compared to the NFKB1 reference sequence (NP_{—}003989). Therefore, NFKB1sv1 polypeptide lacks an internal 33 amino acid region corresponding to the amino acid region encoded by exon 5 as compared to the NFKB1 reference sequence (NP_{—}003989). The NFKB1sv2 polypeptide lacks a 372 base pair region corresponding to exons 17 and 18 of the NFKB1 gene. The deletion of exons 17 and 18 does not disrupt the protein reading frame as compared to the NFKB1 reference sequence (NP_{—}003989). Therefore, NFKB1sv1 polypeptide lacks an internal 124 amino acid region corresponding to the amino acid region encoded by exons 17 and 18 as compared to the NFKB1 reference sequence (NP_{—}003989).

Both the amino acid and nucleic acid sequences of NFKB1sv1 or NFKB1sv2 can be used to help identify and obtain NFKB1sv1 or NFKB1sv2 polypeptides, respectively. For example, SEQ ID NO 3 can be used to produce degenerative nucleic acid probes or primers for identifying and cloning nucleic acid polynucleotides encoding for an NFKB1sv1 polypeptide. In addition, polynucleotides comprising, consisting, or consisting essentially of SEQ ID NO 3 or fragments thereof, can be used under conditions of moderate stringency to identify and clone nucleic acids encoding NFKB1sv1 polypeptides from a variety of different organisms. The same methods can also be performed with polynucleotides comprising, consisting, or consisting essentially of SEQ ID NO 5, or fragments thereof, to identify and clone nucleic acids encoding NFKB1sv2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with NFKB1sv1 or NFKB1sv2 obtained from a particular source, derivatives can be produced. Such derivatives include polypeptides with amino acid substitutions, additions and deletions. Changes to NFKB1sv1 or NFKB1sv2 to produce a derivative having essentially the same properties should be made in a manner not altering the tertiary structure of NFKB1sv1 or NFKB1sv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

NFKB1sv1 and NFKB1sv2 Antibodies

Antibodies recognizing NFKB1sv1 or NFKB1sv2 can be produced using a polypeptide containing SEQ ID NO 4 in the case of NFKB1sv1 or SEQ ID NO 6 in the case of NFKB1sv2, respectively, or a fragment thereof, as an immunogen. Preferably, an NFKB1sv1 polypeptide used as an immunogen consists of a polypeptide of SEQ ID NO 4 or a SEQ ID NO 4 fragment having at least 10 contiguous amino acids in length corresponding to the polynucleotide region representing the junction resulting from the splicing of exon 4 to exon 6 of the NFKB1 gene. Preferably, a NFKB1sv2 polypeptide used as an immunogen consists of a polypeptide derived from SEQ ID NO 6 or a SEQ ID NO 6 fragment, having at least 10 contiguous amino acids in length corresponding to a polynucleotide region representing the junction resulting from the splicing of exon 16 to exon 19 of the NFKB1 gene.

In some embodiments where, for example, NFKB1sv1 polypeptides are used to develop antibodies that bind specifically to NFKB1sv1 and not to other isoforms of NFKB1, the NFKB1sv1 polypeptides comprise at least 10 amino acids of the NFKB1sv1 polypeptide sequence corresponding to a junction polynucleotide region created by the alternative splicing of exon 4 to exon 6 of the primary transcript of the NFKB1 gene (see FIG. 1). For example, the amino acid sequence: amino terminus-EQPKQICNYV-carboxy terminus [SEQ ID NO 9] represents one embodiment of such an inventive NFKB1sv1 polypeptide wherein a first 5 amino acid region is encoded by nucleotide sequence at the 3′ end of exon 4 of the NFKB1 gene and a second 5 amino acid region is encoded by the nucleotide sequence directly after the novel splice junction. Preferably, at least 10 amino acids of the NFKB1sv1 polypeptide comprise a first continuous region of 2 to 8 amino acids that is encoded by nucleotides at the 3′ end of exon 4 and a second continuous region of 2 to 8 amino acids that is encoded by nucleotides at the 5′ end of exon 6.

In other embodiments where, for example, NFKB1sv2 polypeptides are used to develop antibodies that bind specifically to NFKB1sv2 and not to other NFKB1 isoforms, the NFKB1sv2 polypeptides comprise at least 10 amino acids of the NFKB1sv2 polypeptide sequence corresponding to a junction polynucleotide region created by the alternative splicing of exon 16 to exon 19 of the primary transcript of the NFKB1 gene (see FIG. 1). For example, the amino acid sequence: amino terminus-NDLYQGDAHV-carboxy terminus [SEQ ID NO 10], represents one embodiment of such an inventive NFKB1sv2 polypeptide wherein a first 5 amino acid region is encoded by a nucleotide sequence at the 3′ end of exon 16 of the NFKB1 gene and a second 5 amino acid region is encoded by a nucleotide sequence directly after the novel splice junction. Preferably, at least 10 amino acids of the NFKB1sv2 polypeptide comprises a first continuous region of 2 to 8 amino acids that is encoded by nucleotides at the 3′ end of exon 16 and a second continuous region of 2 to 8 amino acids that is encoded by nucleotides at the 5′ end of exon 19.

In other embodiments, NFKB1sv1 -specific antibodies are made using an NFKB1sv1 polypeptide that comprises at least 20, 30, 40 or 50 amino acids of the NFKB1sv1 sequence that corresponds to a junction polynucleotide region created by the alternative splicing of exon 4 to exon 6 of the primary transcript of the NFKB1 gene. In each case the NFKB1sv1 polypeptides are selected to comprise a first continuous region of at least 5 to 15 amino acids that is encoded by nucleotides at the 3′ end of exon 4 and a second continuous region of 5 to 15 amino acids that is encoded by nucleotides directly after the novel splice junction.

In other embodiments, NFKB1sv2-specific antibodies are made using an NFKB1sv2 polypeptide that comprises at least 20, 30, 40 or 50 amino acids of the NFKB1sv2 sequence that corresponds to a junction polynucleotide region created by the alternative splicing of exon 16 to exon 19 of the primary transcript of the NFKB1 gene. In each case the NFKB1sv2 polypeptides are selected to comprise a first continuous region of at least 5 to 15 amino acids that is encoded by nucleotides at the 3′ end of exon 16 and a second continuous region of 5 to 15 amino acids that is encoded by nucleotides directly after the novel splice junction.

Antibodies to NFKB1sv1 or NFKB1sv2 have different uses, such as to identify the presence of NFKB1sv1 or NFKB1sv2, respectively, and to isolate NFKB1sv1 or NFKB1sv2 polypeptides, respectively. Identifying the presence of NFKB1sv1 can be used, for example, to identify cells producing NFKB1sv1. Such identification provides an additional source of NFKB1sv1 and can be used to distinguish cells known to produce NFKB 1sv1 from cells that do not produce NFKB1sv1. For example, antibodies to NFKB1sv1 can distinguish human cells expressing NFKB1sv1 from human cells not expressing NFKB1sv1 or non-human cells (including bacteria) that do not express NFKB1sv1. Such NFKB1sv1 antibodies can also be used to determine the effectiveness of NFKB1sv1 ligands, using techniques well known in the art, to detect and quantify changes in the protein levels of NFKB1sv1 in cellular extracts, and in situ immunostaining of cells and tissues. In addition, the same above-described utilities also exist for NFKB1sv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

NFKB1sv1 and NFKB1sv2 Binding Assay

A number of compounds known to modulate NFKB activity have been disclosed (see for example, Baldwin, 1996, Annu Rev Immunol 14: 649-83; Chen et al., 1999, Clinical Chemistry 45: 7-17; Baldwin, 2001, J Clin Investig 107: 241-246; Barnes and Karin, 1997, New England J of Med 336: 1066-1071). Tepoxalin, a dual inhibitor of cyclooxygenase and 5-lipoxygenase, functions to inhibit NFκB induction in multiple cell types (Kazmi et al., 1995, J. Cell. Biochem. 57: 299-310). Interleukin-10 has been reported to inhibit the induction of NFκB by lipopolysaccharide in human peripheral blood mononuclear cells (Wang et al., 1995, J. Biol Chem 270: 9558-9563). Glucocorticoids, nitric oxide, cyclosporine, rapamycin, and salicylates also act as inhibitors of NFκB activation (reviewed in Baldwin, 1996, Annu Rev Immunol 14: 649-83; Chen et al., 1999, Clinical Chemistry 45: 7-17; Baldwin, 2001, J Clin Investig 107: 241-246; Barnes and Karin, 1997, New England J of Med 336: 1066-1071). In addition, substituted indole inhibitors of NFκB activity have been described (WO 01/30774). Methods for screening compounds for their effects on NFKB1 activity have also been disclosed (see for example WO 01/74900; WO 02/086076). A person skilled in the art may use these methods to screen NFKB1sv1 or NFKB1sv2 polypeptides for compounds that bind to, and in some cases functionally alter, each respective NFKB1 isoform protein.

NFKB1sv1, NFKB1sv2, or fragments thereof, can be used in binding studies to identify compounds binding to or interacting with NFKB1sv1, NFKB1sv2, or fragments thereof, respectively. In one embodiment, NFKB1sv1, or a fragment thereof can be used in binding studies with NFKB1 isoform protein, or a fragment thereof, to identify compounds that: bind to or interact with NFKB1sv1 and other NFKB1 isoforms; bind to or interact with one or more other NFKB1 isoforms and not with NFKB1sv1. A similar series of compound screens can also be performed using NFKB1sv2 rather than, or in addition to, NFKB1sv1. Such binding studies can be performed using different formats including competitive and non-competitive formats. Further competition studies can be carried out using additional compounds determined to bind to NFKB1sv1, NFKB1sv2, or other NFKB1 isoforms.

The particular NFKB1sv1 or NFKB1sv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using NFKB1sv1 expressed from a recombinant nucleic acid. Alternatively, recombinantly expressed NFKB1sv1 consists of the SEQ ID NO 4 amino acid sequence. In addition, binding studies are performed using NFKB1sv2 expressed from a recombinant nucleic acid. Alternatively, recombinantly expressed NFKB1sv2 consists of the SEQ ID NO 6 amino acid sequence.

Binding assays can be performed using individual compounds or preparations containing different numbers of compounds. A preparation containing different numbers of compounds having the ability to bind to NFKB1sv1 or NFKB1sv2 can be divided into smaller groups of compounds that can be tested to identify the compound(s) binding to NFKB1sv1 or NFKB1sv2, respectively.

Binding assays can be performed using recombinantly produced NFKB1sv1 or NFKB1sv2 present in different environments. Such environments include, for example, cell extracts and purified cell extracts containing a NFKB1sv1 or NFKB1sv2 recombinant nucleic acid; and also include, for example, the use of a purified NFKB1sv1 or NFKB31 sv2 polypeptide produced by recombinant means which is introduced into different environments.

In one embodiment of the invention, a binding method is provided for screening for a compound able to bind selectively to NFKB1sv1. The method comprises the steps: providing a NFKB1sv1 polypeptide comprising SEQ ID NO 4; providing a NFKB1 isoform polypeptide that is not NFKB1sv1; contacting the NFKB1sv1 polypeptide and the NFKB1 isoform polypeptide that is not NFKB1sv1 with a test preparation comprising one or more test compounds; and then determining the binding of the test preparation to the NFKB1sv1 polypeptide and to the NFKB1 isoform polypeptide that is not NFKB1sv1, wherein a test preparation that binds to the NFKB1sv1 polypeptide, but does not bind to NFKB1 isoform polypeptide that is not NFKB1sv1, contains one or more compounds that selectively bind to NFKB1sv1.

In one embodiment of the invention, a binding method is provided for screening for a compound able to bind selectively to NFKB1sv2. The method comprises the steps: providing a NFKB1sv2 polypeptide comprising SEQ ID NO 6; providing a NFKB1 isoform polypeptide that is not NFKB1sv2; contacting the NFKB1sv2 polypeptide and the NFKB1 isoform polypeptide that is not NFKB1sv2 with a test preparation comprising one or more test compounds; and then determining the binding of the test preparation to the NFKB1sv2 polypeptide and to the NFKB1 isoform polypeptide that is not NFKB1sv2, wherein a test preparation that binds to the NFKB1sv2 polypeptide, but does not bind to NFKB1 isoform polypeptide that is not NFKB1sv2, contains one or more compounds that selectively binds to NFKB1sv2.

In another embodiment of the invention, a binding method is provided for screening for a compound able to bind selectively to a NFKB1 isoform polypeptide that is not NFKB1sv1. The method comprises the steps: providing a NFKB1sv1 polypeptide comprising SEQ ID NO 4; providing a NFKB1 isoform polypeptide that is not NFKB1sv1; contacting the NFKB1sv1 polypeptide and the NFKB1 isoform polypeptide that is not NFKB1sv1 with a test preparation comprising one or more test compounds; and then determining the binding of the test preparation to the NFKB1sv1 polypeptide and the NFKB1 isoform polypeptide that is not NFKB1sv1, wherein a test preparation that binds the NFKB1 isoform polypeptide that is not NFKB1sv1, but does not bind NFKB1sv1, contains a compound that selectively binds the NFKB1 isoform polypeptide that is not NFKB1sv1. Alternatively, the above method can be used to identify compounds that bind selectively to a NFKB1 isoform polypeptide that is not NFKB1sv2 by performing the method with NFKB1sv2 protein comprising SEQ ID NO 6.

The above-described selective binding assays can also be performed with a polypeptide fragment of NFKB1sv1 or NFKB1sv2, wherein the polypeptide fragment comprises at least 10 consecutive amino acids that are coded by a nucleotide sequence that bridges the junction created by the splicing of the 3′ end of exon 4 to the 5′ end of exon 6 in the case of NFKB1sv1 or by the splicing of the 3′ end of exon 16 to the 5′ end of exon 19, in the case of NFKB1sv2. Similarly, the selective binding assays may also be performed using a polypeptide fragment of an NFKB1 isoform polypeptide that is not NFKB1sv1 or NFKB1sv2, wherein the polypeptide fragment comprises at least 10 consecutive amino acids that are coded by: a) a nucleotide sequence that is contained within exons 5, 17, or 18, of the NFKB1 gene; or b) a nucleotide sequence that bridges the junction created by the splicing of the 3′ end of exon 4 to the 5′ end of exon 5, the splicing of the 3′ end of exon 5 to the 5′ end of exon 6, the splicing of the 3′end of exon 16 to the 5′ end of exon 17, the splicing of the 3′ end of exon 17 to the 5′ end of exon 18, or the splicing of the 3′ end of exon 18 to the 5′ end of exon 19 of the NFKB1 gene.

NFKB1 Functional Assays

NFKB1 encodes p50, an essential component of the NFκB complex that plays an integral role in the cascade leading to the activation of NFκB and the transcription of genes in response to harmful stimuli. NFκB activity also depends on its phosphorylation state. The identification of NFKB1sv1 and NFKB1sv2 as splice variants of NFKB1 provides a means for screening for compounds that bind to NFKB1sv1 and/or NFKB1sv2 protein thereby altering the ability of the NFKB1sv1 and/or NFKB1sv2 polypeptide to be bound by IκB inhibitor proteins, relocate to the nucleus, transcriptionally activate target genes, or be phosphorylated. Assays involving a functional NFKB1sv1 or NFKB1sv2 polypeptide can be employed for different purposes, such as selecting for compounds active at NFKB1sv1 or NFKB1sv2; evaluating the ability of a compound to effect the phosphorylation of each respective splice variant polypeptide; and mapping the activity of different NFKB1sv1 and NFKB1sv2 regions. NFKB1sv1 and NFKB1sv2 activity can be measured using different techniques such as: detecting a change in the intracellular conformation of NFKB1sv1 or NFKB1sv2; detecting a change in the intracellular location of NFKB1sv1 or NFKB1sv2; detecting the amount of binding of NFKB1sv1 or NFKB1sv2 to IKB inhibitors; or by measuring the transcriptional activation activity of the NFκB complex.

Recombinantly expressed NFKB1sv1 and NFKB1sv2 can be used to facilitate the determination of whether a compound's activity in a cell is dependent upon the presence of NFKB1sv1 or NFKB1sv2. For example, NFKB1sv1 can be expressed by an expression vector in a cell line and used in a co-culture growth assay, such as described in U.S. Pat. No. 6,518,035, to identify compounds that alter the growth of the cell expressing NFKB1sv1 from the expression vector as compared to the same cell line but lacking the NFKB1sv1 expression vector. Alternatively, determination of whether a compound's activity on a cell is dependent upon the presence of NFKB1sv1 or NFKB1sv2 can also be done using gene expression profile analysis methods as described, for example, in U.S. Pat. No. 6,324,479. Similar assays can also be used for NFKB1sv2.

Several methods have been used to determine NFκB activation or its function. Electrophoretic mobility shift assays are used to determine the inducible and constitutive NFκB in the nuclei (see for example, Ryan et al., 2000, Nature 404: 892-897; Aljada et al., 1999, J Clin Endocrinol Metab 84: 3386-3389; Digicaylioglu and Lipton, 2001, Nature 412: 641-647). A total nuclear extract is incubated with a α-³²P-labeled double-stranded probe containing a consensus κB-site. The protein bound to the κB probe can be resolved by 5% sodium dodecyl sulfate-polyacrylamide gel electrophoresis and autoradiography. If specific antibodies are included during the incubation of nuclear extract with the κB probe, the member composition of the NFκB complex can also be determined. NFκB nuclear translocation can be quantified by the use of fluorescence-labeled antibodies against different NFκB family members in tissues sections (reviewed in Chen et al., Clin Chemistry 45: 7-17). Patch clamp and flow cytometic methods have also been used to determine the nuclear translocation of NFKB through the nuclear pore complex (Bustamante et al., 1995, J Membrane Biol 146: 253-261; Foulds, S., 1997, Cytometry 29: 182-186). The transcriptional activity of NFκB can be determined by transfecting cells with a reporter construct containing the chloramphenicol acetyltransferase, luciferase, or other reporter gene under the control of κB elements (see for example Digicaylioglu and Lipton, 2001, Nature 412: 641-647; Trompouki et al., 2003, Nature 424: 793-796). Reporter gene activity can be measured by ELISA, thin layer chromatography, a luminometer, or a scintillation counter. Cellular proliferation and apoptotic assays have also been described (Romashkova and Makarov, 1999, Nature, 401: 86-90; Digicaylioglu and Lipton, 2001, Nature 412: 641-647). A variety of other assays has been used to investigate the properties of NFKB1 and therefore would also be applicable to the measurement of NFKB1sv1 or NFKB1sv2 functions.

NFKB1sv1 or NFKB1sv2 functional assays can be performed using cells expressing NFKB1sv1 or NFKB1sv2 at a high level. These proteins will be contacted with individual compounds or preparations containing different compounds. A preparation containing different compounds where one or more compounds affect NFKB1sv1 or NFKB1sv2 in cells over-producing NFKB1sv1 or NFKB1sv2 as compared to control cells containing an expression vector lacking NFKB1sv1 or NFKB1sv2 coding sequences, can be divided into smaller groups of compounds to identify the compound(s) affecting NFKB1sv1 or NFKB1sv2 activity, respectively.

NFKB1sv1 or NFKB1sv2 functional assays can be performed using recombinantly produced NFKB1sv1 or NFKB1sv2 present in different environments. Sguch environments include, for example, cell extracts and purified cell extracts containing NFKB1sv1 or NFKB1 sv2 expressed from recombinant nucleic acid; and the use of purified NFKB1sv1 or NFKB1sv2 produced by recombinant means that is introduced into a different environment suitable for measuring binding or kinase activity.

Modulating NFKB1sv1 and NFKB1sv2 Expression

NFKB1sv1 or NFKB1sv2 expression can be modulated as a means for increasing or decreasing NFKB1sv1 or NFKB1sv2 activity, respectively. Such modulation includes inhibiting the activity of nucleic acids encoding the NFKB1 isoform target to reduce NFKB1 isoform protein or polypeptide expression, or supplying NFKB1 nucleic acids to increase the level of expression of the NFKB1 target polypeptide thereby increasing NFKB1 activity.

Inhibition of NFKB1sv1 and NFKB1sv2 Activity

NFKB1sv1 or NFKB1sv2 nucleic acid activity can be inhibited using nucleic acids recognizing NFKB1sv1 or NFKB1sv2 nucleic acid and affecting the ability of such nucleic acid to be transcribed or translated. Inhibition of NFKB1sv1 or NFKB1sv2 nucleic acid activity can be used, for example, in target validation studies.

A preferred target for inhibiting NFKB1sv1 or NFKB1sv2 is mRNA stability and translation. The ability of NFKB1sv1 or NFKB1sv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can alter nucleic acid stability. Examples of references describing different anti-sense molecules, and ribozymes, and the use of such molecules, are provided in U.S. Pat. Nos. 5,849,902; 5,859,221; 5,852,188; and 5,616,459. Methods for modulating target gene transcription by administering double stranded DNA having a sequence specific for the NFKB transcription factor binding sequence have been described (WO 95/11687). Techniques for reducing the activity of NFκB by delivering anti-sense oligodeoxynucleotides or ribozymes specific for an RNA encoding a protein subunit of NFKB has also been disclosed (WO 01/57061; U.S. Pat. No. 5,591,840; U.S. 2002/0177568). Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc.

Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing NFKB1sv1 and NFKB1sv2 Expression

Nucleic acids encoding NFKB1sv1 or NFKB1sv2 can be used, for example, to cause an increase in NFKB1 activity or to create a test system (e.g., a transgenic animal) for screening for compounds affecting NFKB1sv1 or NFKB1sv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of NFKB1sv1 and NFKB1sv2 Using Microarrays

To identify variants of the “normal” splicing of exon regions encoding NFKB1, an exon junction microarray, comprising probes complementary to each splice junction resulting from splicing of the 24 exon coding sequences in NFKB1 heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least two of the exon junctions of NFKB1 mRNA was altered in some of the tissues examined, indicating the possible presence of NFKB1 splice variant mRNA populations. Reverse transcription and polymerase chain reactions (RT-PCR) were then performed using oligonucleotide primers complementary to exons 4 and 8 or complementary to exons 16 and 20 to confirm the exon junction array results and to allow the sequence structure of the splice variants to be determined.

### Example 2

### Confirmation of NFKB1sv1 and NFKB1sv2 Using RT-PCR

The structure of NFKB1 mRNA in the region corresponding to exons 4 to 8 and exons 16 to 20 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 4 and exon 8 or to sequences in exon 16 and 20 of the reference exon coding sequences in NFKB1 (NM_{—}003998.2). Based upon the nucleotide sequence of NFKB1 mRNA, the NFKB1 exon 4 and exon 8 primer set (hereafter NFKB1₄₋₈ primer set) was expected to amplify a 482 base pair amplicon representing the “reference” NFKB1 mRNA region. The NFKB1 exon 4 forward primer has the sequence: 5′ GGCCCATACCTTCAAATATTAGAGCAACC 3′ [SEQ ID NO 11]; and the NFKB1 exon 8 reverse primer has the sequence: 5′ AGAGCTGCTTGGCGGATTAGCTCTTTTT 3′ [SEQ ID NO 12]. Based upon the nucleotide sequence of NFKB1 mRNA, the NFKB1 exon 16 and exon 20 primer set (hereafter NFKB1₁₆₋₂₀ primer set) was expected to amplify a 647 base pair amplicon representing the “reference” NFKB1 mRNA region. The NFKB1 exon 16 forward primer has the sequence: 5′ TCATCCACCTTCATTCTCAACTTGTGAGG 3′ [SEQ ID NO 13]; and the NFKB1 exon 20 reverse primer has the sequence

| | |
|---|---|
| 5′ ATCCTCTCCTGCATTTTCCCAAGAGTCA 3′. | [SEQ ID NO 14] |

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons were obtained from human mRNA samples using the NFKB1₄₋₈ primer set (data not shown). Every human tissue and cell line assayed except skeletal muscle exhibited the expected amplicon size of 482 base pairs for normally spliced NFKB1 mRNA. However, in addition to the expected NFKB1 amplicon of 482 base pairs, brain-cerebellum, trachea, bone marrow, brain-amygdala, brain-corpus callosum, ileocecum, spleen, melanoma, lung carcinoma, descending colon, brain-hippocampus and heart-interventricular septum also exhibited an amplicon of about 384 base pairs

At least two different RT-PCR amplicons were obtained from human mRNA samples using the NFKB1₁₆₋₂₀ primer set (data not shown). Every human tissue and cell line assayed exhibited the expected amplicon size of 647 base pairs for normally spliced NFKB1 mRNA. However, in addition to the expected NFKB1 amplicon of 647 base pairs, skeletal muscle also exhibited an amplicon of about 276 base pairs. The tissues in which NFKB1sv1 and NFKB1sv2 mRNAs were detected are listed in Table 1 and are marked by an “x”:

TABLE 1
Tissue distribution of NFKB1sv1 and NFKB1sv1 polynucleotides

| Sample | NFKB1sv1 | NFKB1sv2 |
|---|---|---|
| Heart | | |
| Kidney | | |
| Liver | | |
| Brain | | |
| Placenta | | |
| Lung | | |
| Fetal Brain | | |
| Leukemia Promyelocytic (HL-60) | | |
| Adrenal Gland | | |
| Fetal Liver | | |
| Salivary Gland | | |
| Pancreas | | |
| Skeletal Muscle | | x |
| Brain Cerebellum | x | |
| Stomach | | |
| Trachea | x | |
| Thyroid | | |
| Bone Marrow | x | |
| Brain Amygdala | x | |
| Brain Caudate Nucleus | | |
| Brain Corpus Callosum | x | |
| Ileocecum | x | |
| Lymphoma Burkitt's (Raji) | | |
| Spinal Cord | | |
| Lymph Node | | |
| Fetal Kidney | | |
| Uterus | | |
| Spleen | x | |
| Brain Thalamus | | |
| Fetal Lung | | |
| Testis | | |
| Melanoma (G361) | x | |
| Lung Carcinoma (A549) | x | |
| Adrenal Medula, normal | | |
| Brain, Cerebral Cortex, normal; | | |
| Descending Colon, normal | x | |
| Prostate | | |
| Duodenum, normal | | |
| Epididymus, normal | | |
| Brain, Hippocamus, normal | x | |
| Ileum, normal | | |
| Interventricular Septum, normal | x | |
| Jejunum, normal | | |
| Rectum, normal | | |

Sequence analysis of the about 384 base pair amplicon amplified using the NFKB1₄₋₈ primer set revealed that this amplicon form results from the splicing of exon 4 of the NFKB1 hnRNA to exon 6; that is, exon 5 coding sequence is completely absent. Sequence analysis of the about 276 base pair amplicon amplified using the NFKB1₁₆₋₂₀ primer set revealed that this amplicon form results from the splicing of exon 16 of the NFKB1 hnRNA to exon 19; that is, exon 17 and 18 coding sequences are completely absent. Thus, the RT-PCR results confirmed the junction probe microarray data reported in Example 1 which suggested that NFKB1 mRNA is composed of a mixed population of molecules wherein in at least two of the NFKB1 mRNA splice junctions are altered.

### Example 3

### Cloning of NFKB1sv1 and NFKB1sv2

Microarray and RT-PCR data indicate that in addition to the normal NFKB1 reference mRNA sequence, NM_{—}003998.2, encoding NFKB1 protein, NP_{—}003989, two novel splice variant forms of NFKB1 mRNA also exist in many tissues.

Clones having a nucleotide sequence comprising the splice variants identified in Example 2 (hereafter referred to as NFKB1sv1 or NFKB1 sv2) are isolated using a 5′ “forward” NFKBJ primer and a 3′ “reverse” NFKB1 primer, to amplify and clone the entire NFKB1sv1 or NFKB1sv2 mRNA coding sequences, respectively. The same 5′ “forward” primer is designed for isolation of full length clones corresponding to the NFKB1sv1 and NFKB1sv2 splice variants and has the nucleotide sequence of 5′ ATGGCAGAAGATGATCCATATTTGGGAA 3′ [SEQ ID NO 15]. The same 3′ “reverse” primer is designed for isolation of full length clones corresponding to the NFKB1sv1 and NFKB1sv2 splice variants and has the nucleotide sequence of

| | |
|---|---|
| 5′ CTAAATTTTGCCTTCTAGAGGTCCTTCC 3′. | [SEQ ID NO 16] |

RT-PCR

The NFKB1sv1 and NFKB1sv2 cDNA sequences are cloned using a combination of reverse transcription (RT) and polymerase chain reaction (PCR). More specifically, about 25 ng of brain cerebellum or skeletal muscle polyA mRNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the NFKB1 “forward” and “reverse” primers with the brain cerebellum RT reaction to amplify NFKB1sv1 or skeletal muscle RT reaction to amplify NFKB1sv2. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted NFKB1sv1 and NFKB1sv2 mRNAs, products of about 2811 and 2538 base pairs, respectively. The remainder of the 50 μl PCR reactions from fetal brain is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified NFKB1sv1 and NFKB1sv2 RT-PCR products from brain cerebellum and skeletal muscle, respectively, are used in a cloning reaction using the reagents and instructions provided with the TOPO TA cloning kit (Invitrogen, Carlsbad, Calif.). About 2 μl of the cloning reaction is used following the manufacturer's instructions to transform TOP 10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative NFKB1sv1 and NFKB1sv2 clones, respectively are identified and prepared for a PCR reaction to confirm the presence of the expected NFKB1sv1 exon 4 to exon 6 and NFKB1sv2 exon 16 to exon 19 splice variant structures. A 25 μl PCR reaction is performed as described above (RT-PCR section) to detect the presence of NFKB1sv1, except that the reaction includes miniprep DNA from the TOPO TA/NFKB1sv1 ligation as a template. An additional 25 μl PCR reaction is performed as described above (RT-PCR section) to detect the presence of NFKB1sv2 except that the reaction includes miniprep DNA from the TOPO TA/NFKB1sv2 ligation as a template. About 10 μl of each 25 μl PCR reaction is run on a 1% Agarose gel and the DNA bands generated by the PCR reaction are visualized and photographed on a UV light box to determine which minipreps samples have PCR product of the size predicted for the corresponding NFKB1sv1 and NFKB1sv2 splice variant mRNAs. Clones having the NFKB1sv1 structure are identified based upon amplification of an amplicon band of 2811 base pairs, whereas a normal reference NFKB1 clone will give rise to an amplicon band of 2910 base pairs. Clones having the NFKB1sv2 structure are identified based upon amplification of an amplicon band of 2538 base pairs, whereas a normal reference NFKB1 clone would give rise to an amplicon band of 2910 base pairs. DNA sequence analysis of the NFKB1sv1 or NFKB1sv2 cloned DNAs confirm a polynucleotide sequence representing the deletion of exon 5 in the case of NFKB1sv1 or the deletion of exons 17 and 18 in the case of NFKB1sv2.

The polynucleotide sequence of NFKB1sv1 mRNA (SEQ ID NO 3) contains an open reading frame that encodes a NFKB1sv1 protein (SEQ ID NO 4) similar to the reference NFKB1 protein (NP_{—}003989), but lacking amino acids encoded by a 99 base pair region corresponding to exon 5 of the full length coding sequence of the reference NFKB1 mRNA (NM_{—}003998.2). The deletion of the 99 base pair region does not change the protein translation reading frame in comparison to the reference NFKB1 protein reading frame. Therefore, the NFKB1sv1 protein is missing an internal 33 amino acid region as compared to the reference NFKB1 (NP_{—}003989). Exons 4 through 11 encode most of the Rel homology domain that is required for DNA binding, dimerization, and interaction with IκB inhibitors (Heron et al, 1995; reviewed in Baldwin, 1996, Annu Rev Immunol 14: 649-83; Chen et al., 1999, Clinical Chemistry 45: 7-17). Furthermore, exon 5 has been reported to be required for the DNA binding activity of the p50 subunit encoded by NFKB1.

The polynucleotide sequence of NFKB1sv2 mRNA (SEQ ID NO 5) contains an open reading frame that encodes a NFKB1sv2 protein (SEQ ID NO 6) similar to the reference NFKB1 protein (NP_{—}003989), but lacking amino acids encoded by a 372 base pair region corresponding to exons 17 and 18 of the full length coding sequence of reference NFKB1 mRNA (NM_{—}003998.2). The deletion of the 372 base pair region does not change the protein translation reading frame in comparison to the reference NFKB1 protein reading frame. Therefore, the NFKB1sv1 protein is missing an internal 124 amino acid region as compared to the reference NFKB1 (NP_{—}003989). Exons 15 to 21 encode seven complete and one partial ankyrin repeat motifs (Heron et al., 1995). Ankyrin repeats are also found in the IκB family of inhibitor polypeptides. The ankyrin repeats in the p105 polypeptide allow p105 to bind and sequester other Rel-related proteins in the cytoplasm, thereby functioning as a Rel-specific inhibitor (IκB) (Rice et al., 1992, Cell 71: 243-253).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 5, or the complement thereof.

2. The purified nucleic acid of claim 1, wherein said nucleic acid comprises a region encoding SEQ ID NO 6.

3. The purified nucleic acid of claim 1, wherein said nucleotide sequence encodes a polypeptide consisting of SEQ ID NO 6.

4. A purified polypeptide comprising SEQ ID NO 6.

5. The polypeptide of claim 4, wherein said polypeptide consists of SEQ ID NO 6.

6. An expression vector comprising a nucleotide sequence encoding SEQ ID NO 6, wherein said nucleotide sequence is transcriptionally coupled to an exogenous promoter.

7. The expression vector of claim 6, wherein said nucleotide sequence encodes a polypeptide consisting of SEQ ID NO 6.

8. The expression vector of claim 6, wherein said nucleotide sequence comprises SEQ ID NO 5.

9. The expression vector of claim 6, wherein said nucleotide sequence consists of SEQ ID NO 5.

10. A method for screening for a compound able to bind to NFKB1sv2 comprising the steps of:
(a) expressing a polypeptide comprising SEQ ID NO 6 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 6.

15. A method of screening for compounds able to bind selectively to NFKB1sv2 comprising the steps of:
(a) providing a NFKB1sv2 polypeptide comprising SEQ ID NO 6;
(b) providing one or more NFKB1 isoform polypeptides that are not NFKB1sv2;
(c) contacting said NFKB1sv2 polypeptide and said NFKB1 isoform polypeptide that is not NFKB1sv2 with a test preparation comprising one or more compounds; and
(d) determining the binding of said test preparation to said NFKB1sv2 polypeptide and to said NFKB1 isoform polypeptide that is not NFKB1sv2, wherein a test preparation that binds to said NFKB1sv2 polypeptide, but does not bind to said NFKB1 polypeptide that is not NFKB1sv2, contains a compound that selectively binds said NFKB1sv2 polypeptide.

16. The method of claim 15, wherein said NFKB1sv2 polypeptide is obtained by expression of said polypeptide from an expression vector comprising a polynucleotide encoding SEQ ID NO 6.

17. The method of claim 16, wherein said polypeptide consists of SEQ ID NO6.

18. A method for screening for a compound able to bind to or interact with a NFKB1sv2 protein or a fragment thereof comprising the steps of:
(a) expressing a NFKB1sv2 polypeptide comprising SEQ ID NO 6 or fragment thereof from a recombinant nucleic acid;
(b) providing to said polypeptide a labeled NFKB1 ligand that binds to said polypeptide and a test preparation comprising one or more compounds; and
(c) measuring the effect of said test preparation on binding of said labeled NFKB1 ligand to said polypeptide, wherein a test preparation that alters the binding of said labeled NFKB1 ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell.

21. The method of claim 18, wherein said polypeptide is expressed from an expression vector.

22. The method of claim 18, wherein said NFKB1sv2 ligand is an NFKB1 inhibitor.

23. The method of claim 21, wherein said expression vector comprises SEQ ID NO 5 or a fragment of SEQ ID NO 5.

24. The method of claim 21, wherein said polypeptide comprises SEQ ID NO 6 or a fragment of SEQ ID NO 6.


SEQUENCE LISTING (16 sequences)

SEQ ID NO: 1 - DNA, 40 bp, Homo sapiens
tattagagcaacctaaacagatctgcaactatgtgggacc

SEQ ID NO: 2 - DNA, 40 bp, Homo sapiens
tgagaaatgatctgtaccagggtgatgcccatgtggacag

SEQ ID NO: 3 - DNA, 2811 bp, Homo sapiens
atggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacacgggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 4 - protein, 936 aa, Homo sapiens
MAEDDPYLGRPEQMFHLDPSLTHTIFNPEVFQPQMALPTADGPYLQILEQPKQICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDTGVETSFRKLSFTESLTSGASLLTLNKMPHDYGQEGPLEGKI

SEQ ID NO: 5 - DNA, 2538 bp, Homo sapiens
atggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacacgggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 6 - protein, 845 aa, Homo sapiens
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDTGVETSFRKLSFTESLTSGASLLTLNKMPHDYGQEGPLEGKI

SEQ ID NO: 7 - DNA, 20 bp, Homo sapiens
acctaaacagatctgcaact

SEQ ID NO: 8 - DNA, 20 bp, Homo sapiens
tctgtaccagggtgatgccc

SEQ ID NO: 9 - protein, 10 aa, Homo sapiens
EQPKQICNYV

SEQ ID NO: 10 - protein, 10 aa, Homo sapiens
NDLYQGDAHV

SEQ ID NO: 11 - DNA, 29 bp, Homo sapiens
ggcccataccttcaaatattagagcaacc

SEQ ID NO: 12 - DNA, 28 bp, Homo sapiens
agagctgcttggcggattagctcttttt

SEQ ID NO: 13 - DNA, 29 bp, Homo sapiens
tcatccaccttcattctcaacttgtgagg

SEQ ID NO: 14 - DNA, 28 bp, Homo sapiens
atcctctcctgcattttcccaagagtca

SEQ ID NO: 15 - DNA, 28 bp, Homo sapiens
atggcagaagatgatccatatttgggaa

SEQ ID NO: 16 - DNA, 28 bp, Homo sapiens
ctaaattttgccttctagaggtccttcc